(19) United States
(12) Patent Application Publication
(10) Pub. No.: US 2006/0286032 A1
(43) Pub. Date: Dec. 21, 2006

(54) A radiosotope-chitosan complex for treatment of prostate cancer

(76) Inventors: Jei-Man Ryu, Kyunggi-do (KR); Dong-Hyuk Shin, Kyunggi-do (KR); Seung-Kyoo Seong, Kyunggi-do (KR); Hee-Bog Yang, Kyunggi-so (KR); Eun-Jung Bae, Seoul (KR); Young-Jun Song, Seoul (KR); Kyung-Bae Park, Taejeon-si (KR); Byung-Chul Shin, Taejeon-si (KR)

Correspondence Address: LUCAS & MERCANTI, LLP, 475 PARK AVENUE SOUTH, 15TH FLOOR, NEW YORK, NY, 10016, US

(21) Appl. No.: 10/570,295

(22) PCT Filed: Sep. 8, 2004
(86) PCT No.: PCT/KR04/02283

§ 371 Date: Feb. 28, 2006

(30) Foreign Application Priority Data
Sep. 8, 2003 (KR) 10-2003-0062771

(51) Int. Cl. A61K 51/00; C08B 37/08

(52) U.S. Cl. 424/1.69; 536/20; 514/55

(57) ABSTRACT

Disclosed is a composition for treating prostate cancer. The composition for treating prostate cancer comprises as an effective ingredient a radioisotope-chitosan complex that includes a therapeutic radioisotope emitting beta radiation and chitosan. Also, the present invention discloses a kit for preparing the composition. When directly administered to a prostate cancer tissue, the radioisotope- chitosan complex is deposited in the applied target site while not leaking from the applied target site, and strongly inhibits the growth of prostate cancer cells while minimizing the side effects of conventional therapies, including urinary incontinence, urethral stricture and rectal bleeding. In addition, the radioisotope-chitosan complex may be used as an effective therapeutic agent for hormone-independent prostate cancer that is resistant to hormone therapy.

### TECHNICAL FIELD

The present invention relates, in general, to a composition for treating prostate cancer comprising a radioisotope-chitosan complex as an effective ingredient and a kit for preparing such a composition. More particularly, the present invention relates to a composition for treating prostate cancer comprising as an effective ingredient a radioisotope-chitosan complex containing a therapeutic radioisotope emitting beta radiation and chitosan, and a kit for preparing such a composition.

### BACKGROUND ART

Prostate cancer is the most common neoplasm among men in western countries, which occurs in about 30% of males over the age 50 and about 80% of males over the age 80, and is the leading cause of death in males. In Korea, the incidence of prostate cancer is relatively low, about 1.2%, but is gradually increasing due to the Westernization of eating habits and the aging society.

The treatment options for prostate cancer include radical prostatectomy involving the surgical removal of the entire prostate gland for removing the cancer, hormone therapy involving the inhibition of production or action of male hormones, chemotherapy using anticancer drugs, and radiation therapy using high-energy rays to lead to the necrosis of cancer cells. Radical prostatectomy is very clinically effective, but carries late complications including urinary incontinence, impotence and urethral stricture. In particular, impotence is caused because the nerves are severed during the surgical operation, and thus, there is no effective therapy for impotence. Hormone therapy is also very clinically effective but cannot be applied to hormone-independent prostate cancer or hormone-resistant prostate cancer. Radiation therapy can be one of two types: external radiation therapy that involves delivering radiation from a source outside the body to a cancer site; and internal radiation therapy that involves injecting a radioactive substance into a cancerous tissue. External beam radiation therapy has complications including rectal bleeding and impotence because the external beam radiation is irradiated even to the bladder and the rectum that are the major organs surrounding the prostate gland.

Internal radiation therapy, directly injecting a radioactive substance into a cancerous tissue, is an advanced treatment method because it can administer a large quantity of radiation only to cancerous tissues and thus has higher therapeutic effects with fewer side effects than the external type or radical prostatectomy.

However, when cancer is to be treated by injecting a radioactive substance into the body and generating radiation therein, an injected radionuclide should be deposited only in cancerous tissues and should not leak to the surrounding area. When an injected radionuclide leaks from an applied cancerous site, it can damage nearby organs, as well as spread through the blood stream to other parts of the body, resulting in widespread damage throughout the body, particularly, lethal damage to the bone marrow. At present, internal radiation therapy for prostate cancer treatment mainly uses radioactive substances such as iodine-125 (I¹²⁵), palladium-103 (Pd¹⁰³) and iridium-192 (Ir¹⁹²). To prevent these radioactive substances (radioisotopes) from leaking to nearby tissues or a surgeon from being exposed to radiation, the radioactive substances are injected using a specific auxiliary apparatus or in sealed seeds. The currently available internal radiation therapy has limited practical applications despite its excellent therapeutic efficacy because it requires high cost and extra space for its application.

Korean Pat. No. 190957 discloses a chelate complex of chitosan and a radionuclide that emits both high-energy beta radiation and low-energy gamma radiation (hereinafter, the complex is referred to as radioisotope-chitosan complex), and the use of the radioisotope-chitosan complex as a therapeutic agent against liver cancer and rheumatoid arthritis.

Based on the background, the present inventors found that the radioisotope-chitosan complex has an excellent therapeutic effect against prostate cancer in addition to liver cancer, thereby leading to the present invention.

### DISCLOSURE

[Technical Problem]

It is therefore an object of the present invention to provide a novel composition for treating prostate cancer, which minimizes the side effects of the conventional therapies, including urinary incontinence, impotence, urethral stricture and rectal bleeding, reduces the time of a related surgical operation and has excellent therapeutic efficacy.

[Technical Solution]

In order to accomplish the above object, the present invention provides a composition for treating prostate cancer comprising a radioisotope-chitosan complex as an effective ingredient.

In addition, the present invention provides a kit for preparing a radioisotope-chitosan complex.

[Advantageous Effects]

The present invention involves the use of a radioisotope-chitosan complex for treating prostate cancer, which may be supplied as it is or as a kit. When directly administered to a prostate cancer tissue, the radioisotope-chitosan complex is deposited in the applied target site while not leaking from the applied target -site, and strongly inhibits the growth of prostate cancer cells while minimizing the side effects of conventional therapies, including urinary incontinence, urethral stricture and rectal bleeding. In addition, the radioisotope-chitosan complex may be used as an effective therapeutic agent for hormone-independent prostate cancer that is resistant to hormone therapy.

### DESCRIPTION OF DRAWINGS

FIG. 1 shows histopathological observations of various tissues after 1 mCi of a complex solution is administered into the prostate gland of rats (hematoxylin & eosin staining; microscopic magnification: ×40).

FIG. 2 shows histopathological observations of AIT tumor transplanted into the prostate gland of rats four weeks after a complex is administered to the rats (hematoxylin & eosin staining). A and B are the prostate of control group. Tumor cells invaded all parenchyma of prostate gland (P) and tumor cells into ductus deference (D) are seen. C and D are the prostate of the group administered with 0.5 mCi of a complex solution, broad necrosis are observed in the center of injection site (arrow) and all of tumor cells in injection site are necrotized. Intact tumor cell is not observed.

### BEST MODEL

### I. Radioisotope-Chitosan Complex

The radioisotope-chitosan complex, which is to be used for treating prostate cancer in the present invention, comprises a therapeutic radioisotope and chitosan as a carrier therefor. The therapeutic radioisotope should not leak from an applied target site, and, after the radioisotope is decayed, a decayed product should be excreted from the body.

1. Therapeutic Radioisotopes

Radionuclides useful in the present invention include all radionuclides including beta ray-emitting radionuclides if they can be applied to the body for therapeutic purposes. Radionuclides may include, in addition to nuclides emitting pure beta rays, gamma ray-emitting nuclides and nuclides emitting both beta and gamma rays.

Preferred radionuclides useful in the present invention are nuclides emitting beta radiation that include ¹⁶⁹Er, ¹⁶⁶Ho, ¹⁵³Sm and ¹⁶⁵Dy and may be determined according to the therapeutic purpose. The beta emitters such as ¹⁶⁹Er, ¹⁶⁶Ho, ¹⁵³Sm and ¹⁶⁵Dy have excellent effects in killing cancer cells by emitting a large quantity of high-energy beta radiation but have weak penetration capability and have a mean penetration range of only 2.2 mm in tissues, resulting in a reduction in the exposure risk of surrounding organs to radiation. In addition, since these beta emitters emit a small quantity of low-energy gamma radiation, their behavior after being injected is easily captured as images. By virtue of these advantages, the beta emitters are suitable for internal radiation therapy.

In particular, holmium-166 (¹⁶⁶Ho) is preferred. Holmium-166 (¹⁶⁶Ho) is a lanthanide element, which is easily formed from neutron activation of naturally occurring holmium-165 (¹⁶⁵Ho) by causing a nuclear reaction by neutron bombardment in an atomic reactor for producing radioisotopes. Holmium-166 has a short half-life of 26.8 hrs and mainly emits beta radiation having an excellent effect on killing cancer cells, as well as emitting a small quantity of gamma radiation and so has only a mean range of 1.23 mm, thereby treating the cancer with a minimum of damage to surrounding tissues or cells. Holmium-166 undergoes beta decay and finally turns into a stable nuclide, holmium-165 (¹⁶⁵Ho). The energy of beta radiation emitted during the beta decay kills cancer cells, and the energy of emitted gamma radiation is utilized for monitoring an injected site or the behavior of holmium-165 (¹⁶⁵Ho), such as radioactivity leakage, as images.

2. Carrier

A carrier for a radionuclide, useful in the present invention, is highly biodegradable, biocompatible chitosan.

Chitosan is a polymer of 2-deoxy-2-amino-D-glucose, which is produced by hydrolysis of chitin that is a major component of crab shells, lobster shells, cuttlebones, and the like. Unlike chitin, chitosan has free amine groups that form chelate complexes with various metal cations. In addition, chitosan exists in a sol phase in an acidic environment, and, as the pH increases, undergoes a phase transition to a gel phase and is eventually transformed into a particle. Due to its properties of being very low toxic and highly biodegradable and biocompatible, chitosan is suitable as a carrier.

Chitosan useful in the present invention includes chitosans having molecular weights of about 100,000 to 1,500,000. The use of a chitosan having a molecular weight of less than 100,000 results in very low labeling yields. When the chitosan has a molecular weight more than 1,500,000, a prepared complex solution is highly viscous and thus difficult to inject. Preferred are chitosans having molecular weights of 400,000 to 1,300,000.

In addition to chitosans, various chitosan derivatives may be used in the present invention.

3. Radioisotope-Chitosan Complex

The radioisotope-chitosan complex of the present invention, which is a chelate complex produced by reacting the chitosan with the therapeutic radioisotope, is very stable in vitro and in vivo because the radioisotope tightly binds to free amine groups of the chitosan. Also, the complex exists in a sol phase in a pH of less than 4.0, and, when injected into the body and meeting body fluids, becomes increasingly viscous and gelated. Thus, the complex in a sol phase may be directly administered to the prostate cancer, and, after being administered, may be gelated in the prostate cancer and selectively deposited only in the prostate cancer, thereby preventing radioactivity leakage from an administration site to the whole body. In particular, since the radioisotope-chitosan complex of the present invention is present in a sol phase in acidic environments and thus administrable in a sol phase, it is evenly distributed in the applied cancer.

4. Kit for Preparing a Radioisotope-Chitosan Complex

The kit of the radioisotope-chitosan complex for treating prostate cancer according to the present invention is a kit for preparing a in-situ product, which comprises a reagent-A(kit-A) containing an aqueous solution of a radioisotope and a reagent-B(kit-B) containing a chitosan solution. The two reagents of the kit may be mixed and dissolved immediately before use and directly injected into the prostate cancer using a syringe, and display therapeutic effects in the cancer. The radioisotope is contained in the reagent A of the kit in a final concentration of 0.5-150 mCi.

### II. Preparation Methods of the Radioisotope-Chitosan Complex and the Kit for Preparing the Radioisotope-Chitosan Complex

1. Preparation Method for the Radioisotope-Chitosan Complex

The radioisotope-chitosan complex is prepared according to the same method as described in Korean Pat. No. 190957. A detailed description of the method follows.

A radioisotope-chitosan complex may be produced by adding a radionuclide solution to a chitosan solution. Since chitosan is well dissolved in an acidic solution, existing in a gel state in a neutral solution and precipitating in an alkali solution, the chitosan solution may be prepared by dissolving chitosan in an acidic solution. An acid useful in the preparation of the chitosan solution is any weak acid, and most preferred acids are carboxylic acids such as acetic acid and formic acid.

A radionuclide solution may be prepared by dissolving a radionuclide-containing compound in water. Examples of the radionuclide-containing compound include nitrates, such as ¹⁶⁵Dy(NO₃)₃ and ¹⁶⁶Ho(NO₃)₃, and chlorides, such as ¹⁶⁵DyCl₃ and ¹⁶⁶HoCl₃. That is, the radionuclide solution is prepared by irradiating neutrons to a solid preparation of an oxide or nitrate of a stable nuclide such as ¹⁶⁴Dy or ¹⁶⁵Ho in an atomic reactor to generate a radionuclide such as ¹⁶⁵Dy or ¹⁶⁶Ho and dissolving the radionuclide in water.

The chitosan solution, which is prepared by dissolving chitosan in an acid, may further comprise a commonly used additive, which is exemplified by a pH controller, an isotonic adjusting agent (e.g., NaCl), a preservative (e.g., benzyl alcohol) and a stabilizer. Ascorbic acid may be used as a stabilizer.

The radionculide solution and the chitosan solution, which are prepared separately as described above, are mixed to provide a radioisotope-chitosan complex solution that emits radiation. Herein, the pH of the reaction mixture preferably ranges from 2.5 to 3.5.

Alternatively, the chitosan solution supplemented with a stabilizer is freeze-dried to provide chitosan powder, and the chitosan powder is added to the radionuclide solution to generate a radioisotope-chitosan complex.

2. Preparation Method for the Kit for Preparing the Radioisotope-Chitosan Complex

The kit for preparing the radioisotope-chitosan complex is prepared according to the following method.

The kit is an in-situ product that comprises a reagent-A consisting of a radionuclide solution and a reagent-B consisting of a chitosan solution. The two reagents of the kit may be mixed and dissolved immediately before use and directly injected using a syringe into a cancerous site or another target site according to the therapeutic purpose, and display therapeutic effects in the injected site.

That is, the chitosan solution and the radionuclide solution are separately prepared, supplied as a kit, and mixed immediately before administration to patients. After the chitosan solution is mixed with the radionuclide solution, the mixture is allowed to stand under gravity for about 10 min to form a radioisotope-chitosan complex.

(1) Preparation Method for Reagent-A

The reagent-A that consists of a radionuclide solution is prepared by dissolving in water a radionuclide-containing compound, which is exemplified by nitrates, such as ¹⁶⁵Dy(NO₃)₃ and ¹⁶⁶Ho (NO₃)₃, and chlorides, such as ¹⁶⁵DyCl₃ and ¹⁶⁶HoCl₃. That is, the radionuclide solution is prepared by irradiating neutrons to a solid preparation of an oxide or nitrate of a stable nuclide such as ¹⁶⁴Dy or ¹⁶⁵Ho in an atomic reactor to generate a radionuclide such as ¹⁶⁵Dy or ¹⁶⁶Ho and dissolving the radionuclide in water. The radioisotope is contained in the reagent A of the kit in a final concentration of 0.5-150 mCi.

(2) Preparation Method for the Reagent-B

The reagent-B that consists of a chitosan solution is prepared by dissolving chitosan in a solution of a weak acid, which is exemplified by carboxylic acids such as acetic acid and formic acid. Also, the chitosan solution may be freeze-dried to be provided in a freeze-dried form. Upon the preparation of the chitosan solution, a commonly used additive may be added to the chitosan solution, which is exemplified by a pH controller, an isotonic adjusting agent (e.g., NaCl), a preservative (e.g., benzyl alcohol) and a stabilizer. Ascorbic acid may be used as a stabilizer.

Immediately before the radioisotope-chitosan complex kit of the present invention is used, the two reagents A and B are mixed and dissolved, and the mixture is allowed to stand under gravity for about 10 min to form a radioisotope-chitosan complex. In this regard, the following description of the radioisotope-chitosan complex of the present invention is applied for the radioisotope-chitosan complex kit of the present invention.

### III. The Use of the Radioisotope-Chitosan Complex as a Composition for Treating Prostate Cancer

1. The Therapeutic Effect of the Radioisotope-Chitosan Complex on Prostate Cancer

(1) In vivo Pharmacokinetics of the Radioisotope-Chitosan Complex

The radioisotope-chitosan complex is injected into the prostate gland of a normal animal and observed for its tissue distribution in various organs to determine whether it has side effects in surrounding tissues and organs. Six hours after administration to the prostate gland, the radioisotope-chitosan complex is present in high concentrations in the administration site, while rarely detected in other major organs including the liver, spleen and kidney (Table 1). In addition, a radioisotope solution not containing chitosan, 24 hrs after being administered, deposits only 30% of its radioactivity in the prostate gland. By contrast, when the radioisotope-chitosan complex of the present invention is administered, almost 90% of its radioactivity is retained in the prostate gland (Table 2). These results indicate that the administered radioisotope-chitosan complex is only slightly leaked to other organs through the blood stream but is deposited only in the prostate gland where the complex has been administered. In addition, for a period of 72 hrs after the radioisotope-chitosan complex was administered, cumulative urinary and fecal excretions of the administered radioactivity were as low as 0.35% and 0.11%, respectively, while 99.19% of the administered radioactivity was retained in the prostate gland. These results indicate that the majority of the administered complex was present in the prostate gland for 72 hrs (Table 5).

(2) Evaluation of the Degree of Radiation Exposure to Surrounding Organs

After the radioisotope-chitosan complex of the present invention is administered, radioactive concentrations were measured in various tissues. Radioactivity was rarely detected in major organs including the brain, thymus, heart, lung, adrenal gland and spleen, as well as in organs adjacent to the prostate gland, which include the bladder, rectum, testes and epididymis. Even after 144 hrs of the administration, more than 98% of the radioactivity was still retained in the prostate gland where the complex had been administered (Tables 3 and 4). These results indicate that the administered radioisotope-chitosan complex was deposited in the prostate gland, and that its radioactivity was only slightly distributed in adjacent organs.

(3) Therapeutic Effect of the Radioisotope-Chitosan Complex on Prostate Cancer

The radioisotope-chitosan complex of the present invention strongly suppresses tumor growth when administered to a cancerous site of an experimental animal to which prostate cancer has been transplanted.

In an animal test using a subcutaneous tumor model, when the radioisotope-chitosan complex of the present invention was administered to a human hormone-independent prostate cancer (DU-145 carcinoma cell line) that rarely produced prostate-specific antigens, it displayed an excellent inhibitory activity against the tumor growth, higher than 95% (Table 6). Also, the present complex had an excellent inhibitory activity against the tumor growth, higher than 90% when administrated to androgen-independent prostate cancer (AIT carcinoma cell line) (Table 7).

In addition, in a test using an orthotopic tumor model in which androgen-independent prostate carcinomas was directly transplanted into the prostate gland of noble rats, two weeks after administration of the present complex, the tumor growth was suppressed, and, four weeks after the administration of the present complex, an inhibitory effect of higher than 90% was observed on the tumor growth, similar to that in the subcutaneous tumor model (Table 8).

These results indicate that the radioisotope-chitosan complex of the present invention has an excellent therapeutic effect against hormone-independent prostate cancer that is resistant to hormone therapy.

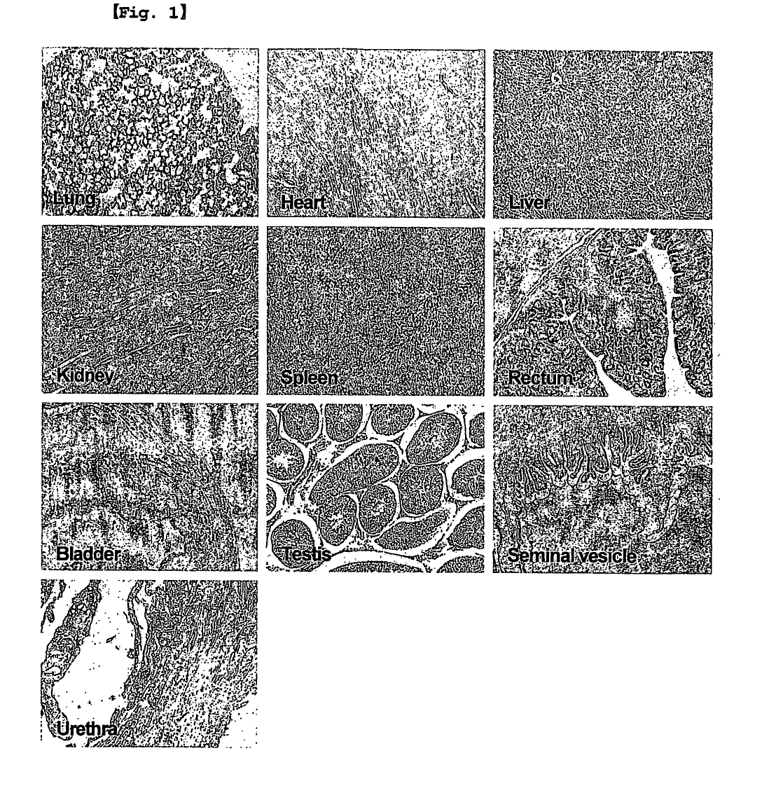

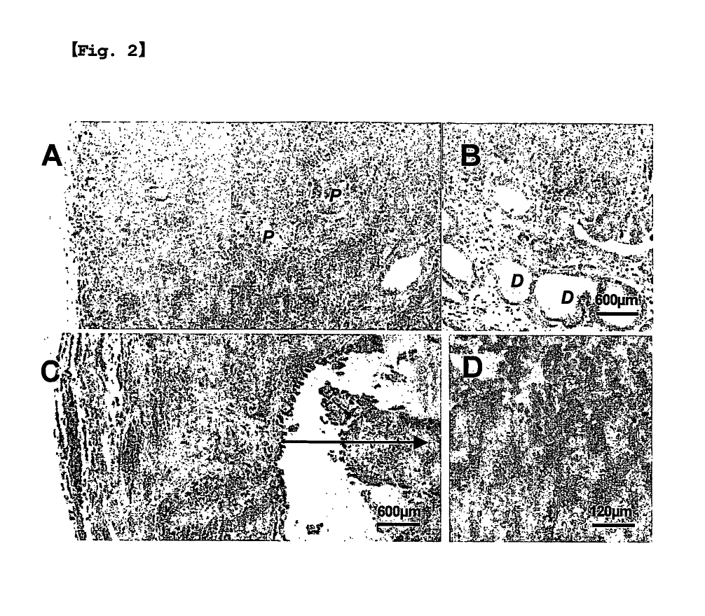

In addition, when, from an experimental animal administered with the radioisotope-chitosan complex of the present invention into the prostate, the heart, lung, liver, kidney, spleen, testes, epididymis, seminal vesicle, bladder, large intestine, rectum and tumor tissues are excised and observed, particular histopathological changes are not found in the organs except for the tumor tissue (FIG. 1). The cellularity of the bone marrow and peripheral blood is not reduced, but a large necrosis is observed in a center of an administration site of the tumor tissue (FIG. 2). These results indicate that the present complex locally acts only in a cancerous site where the complex has been administered and induces the necrosis of cancer cells therein.

Taken together, the action mechanism of the radioisotope-chitosan complex of the present invention in treating prostate cancer is as follows. The radioisotope-chitosan complex of the present invention exists in a sol phase in an acidic solution of a pH less than 4. When the present complex is exposed to body fluids upon administration into the body and thus has an increased pH value, it undergoes a phase transition to a gel phase and becomes less fluidic. Due to this property of the present complex, the administered radionuclide is distributed in the prostate cancer that is an administration site, and kills cancer cells therein by emitting beta radiation while retained in the cancerous site for a long period of time with no risk of leakage to other organs, thereby increasing the necrosis of prostate carcinomas and minimizing side effects in normal tissues. In addition, the present complex has an excellent therapeutic effect against hormone-independent prostate cancer that is resistant to hormone therapy.

2. Administration Method

The radioisotope-chitosan complex of the present invention may be administered in an injectable formulation. For parenteral administration, the present complex may be formulated into various pharmaceutical preparations, for example, sterile aqueous or non-aqueous solutions, dispersions, suspensions, emulsions, and sterile powder capable of being formulated into sterile solutions or suspensions immediately before use. Examples of suitable sterile aqueous and non-aqueous carriers, diluting agents, solvents or vehicles include water, physiological saline, ethanol, polyols (e.g., glycerol, propylene glycol, polyethylene glycol, etc.) and suitable mixtures thereof, vegetable oils (e.g., olive oil), and injectable organic esters (e.g., ethyloleate). For example, the dispersions and suspensions may be maintained at a suitable size using a coating material such as lecithin and may be maintained at a suitable fluidity using a surfactant. Parenteral compositions may include auxiliary agents, such as antiseptics, humectants, emulsifiers and dispersing agents. Sterilization of injectable preparations may be achieved, for example, by filtering the preparations through a sterile filter, or by sterilizing in advance components for preparing a mixture before mixing, upon preparation or immediately before administration (for example, in the case that the preparation is provided in the form of a double-container injection package).

3. Effective Amount

In the case of containing ¹⁶⁶Ho, the radioisotope-chitosan complex of the present invention may be administered in a dose of 0.5-150 mCi or in an amount of 0.5-50 mCi per cm³ tumor according to the type of disease and the size of target sites.

### MODE FOR INVENTION

A better understanding of the present invention may be obtained through the following examples or experimental examples which are set forth to illustrate, but are not to be construed as the limit of the present invention.

### EXAMPLE 1

### Preparation of a Holmium-166 (¹⁶⁶Ho)-Chitosan Complex

1) Preparation of a 10% ¹⁶⁶Ho(NO₃)₃.5H₂O Solution

200 mg of a labeling material, ¹⁶⁵Ho(NO₃)₃.5H₂O, was placed into a polyethylene tube, and neutrons were irradiated from an irradiation hole for 50 hrs at a thermal neutron flux of 4.0×10¹³n/cm²sec in an atomic reactor (Hanaro in Korea) using a pneumatic tube set. The produced ¹⁶⁶Ho(NO₃)₃.5H₂O was dissolved in distilled water.

2) Preparation of a Chitosan Solution

10 g of chitosan (molecular weight: about 500,000; hydrolysis degree: 85%) was added to 1 L of a 1% acetic acid solution and completely dissolved with stirring. 2 ml of the resulting chitosan solution were aliquotted into 10 ml glass vials.

3) Preparation of a ¹⁶⁶Ho-Chitosan Complex

0.1 ml of the 10% ¹⁶⁶Ho(NO₃)₃.5H₂O solution as prepared above was mixed with the chitosan solution at room temperature and allowed to stand at room temperature for over 10 min, thus generating a ¹⁶⁶Ho-chitosan complex.

### EXAMPLE 2

### Preparation of a Kit for Preparing a Holmium-166 (166Ho)-Chitosan Complex

1) Preparation of Reagent-A (Radionuclide Solution)

A radionuclide solution was prepared according to the same method as in Example 1 except that a final concentration of a radionuclide was 20 mCi/ml and prepared as 1-ml aliquots.

2) Preparation of a Reagent-B (Freeze-Dried Chitosan)

20 mg of chitosan and 15 mg of ascorbic acid were dissolved in 2 ml 1% acetic acid, adjusted to a pH of 3.0 using 0.5 N HCl and sterilized using a filter. Then, the filtrate was freeze-dried and stored at 4° C.

3) Use of the Kit

For use of the kit, the reagent-A was mixed with the reagent-B and allowed to stand at room temperature for about 10 min to form a radionuclide-chitosan complex, thus generating a ¹⁶⁶Ho-chitosan complex solutoin. In the following Experimental Examples, the ¹⁶⁶Ho-chitosan complex solution (hereinafter, referred to simply as “complex solution”), prepared using the kit for preparing a ¹⁶⁶Ho-chitosan complex as prepared above, was used.

### EXPERIMENTAL EXAMPLE 1

After one week acclimation, male SD rats were anesthetized with diethylether, and an about 2-cm midline incision was made in the lower abdomen of the pubis. After the prostate gland was exposed along with the bladder, 25 μl and 50 μl (each about 100 μCi) of the complex solution prepared in Example 2 were individually administered to the prostate gland using a syringe. After the rats were sacrificed 0.5 hr and 6 hrs after administration, cryosections were prepared, and whole-body autoradiography was carried out to measure radioactive concentrations in the liver, spleen, kidney, the end region of the femur, spine and administration sites. The measured radioactive concentrations (% ID/g) in various organs after injection of the complex solution are given in Table 1, below.

Numerals of Table 1 indicate mean+standard deviation (n=3).

TABLE 1
Radioactive concentrations (% ID/g) in various organs after
injection of the complex solution

| | Radioactive concentrations (% ID/g)* | | | |
|---|---|---|---|---|
| | 25 μl administration | | 50 μl administration | |
| Organs | 30 min | 6 hrs | 30 min | 6 hrs |
| Administration site | 4258 ± 732.9 | 4478 ± 1212.4 | 3196 ± 212.5 | 3054 ± 1873.0 |
| Liver | ND^{a} | 0.04 ± 0.015 | 0.01 ± 0.007 | 0.03 ± 0.008 |
| Spleen | ND | 0.03 ± 0.007 | ND | 0.02 ± 0.014 |
| Kidney | ND | 0.01 ± 0.002 | 0.02 ± 0.011 | 0.01 ± 0.001 |
| End region of femur | 0.05 ± 0.030 | 0.16 ± 0.047 | 0.05 ± 0.030 | 0.10 ± 0.065 |
| Spine | 0.02 ± 0.009 | 0.04 ± 0.011 | 0.02 ± 0.007 | 0.04 ± 0.018 |

^{a} not detected.
*% ID/g = [radioactive concentration in an organ (PSL/weight of an organ (g))/administered radioactive concentration (PSL)] × 100

As shown in Table 1, even 6 hrs after administration, over 90% of radioactivity of the complex solution was detected in the administration site, whereas no radioactivity was detected in major organs including the liver, spleen and kidney. The leakage of radioactivity from the administration of the complex solution to the other organs was not correlated with the administration amount.

### EXPERIMENTAL EXAMPLE 2

To investigate advantages of the ¹⁶⁶Ho-chitosan complex, autoradiography was carried out for a group administered with the complex solution and a control group administered with a ¹⁶⁶Ho(NO₃)₃.5H₂O solution not containing chitosan. In the complex solution administration group, 25 μl of the complex solution was administered, which was equal to about 100 μCi. The control group was administered with a ¹⁶⁶Ho(NO₃)₃.5H₂O solution not containing chitosan with the same dosage and radioactive concentration as in the complex solution administration group. This test was carried out according to the same method as in Experimental Example 1, and each test drug was administered to the prostate gland. After 0.5, 2, 6 and 24 hrs, rats were sacrificed and evaluated by autoradiography. The measured radioactive concentrations (% ID/g) in various organs after injection of the complex solution and the ¹⁶⁶Ho(NO₃)₃.5H₂O solution are given in Table 2, below.

TABLE 2
Radioactive concentrations in various organs after injection
of the complex solution and the ¹⁶⁶Ho(NO₃)₃.5H₂O solution

| | Radioactive concentrations after administration of test | | | |
|---|---|---|---|---|
| | drugs, % ID/g* | | | |
| Test groups and organs | 30 min | 2 hrs | 6 hrs | 24 hrs |
| Complex administration group | | | | |
| Administration site | 4149 ± 1563.2 | 3246 ± 764.0 | 4815 ± 1177.4 | 3998 ± 705.1 |
| Liver | 0.01 ± 0.004 | 0.016 | 0.04 ± 0.024 | 0.04 ± 0.004 |
| Spleen | ND^{a} | ND | 0.03 ± 0.027 | 0.04 ± 0.010 |
| Kidney | ND | ND | 0.02^{b} | 0.03 ± 0.016 |
| End region of femur | 0.04 ± 0.015 | 0.07 ± 0.013 | 0.21 ± 0.101 | 0.55 ± 0.324 |
| Spine | 0.01 ± 0.002 | 0.02 ± 0.004 | 0.05 ± 0.024 | 0.14 ± 0.070 |
| ¹⁶⁶Ho administration group | | | | |
| Administration site | 4532 ± 513.4 | 4404 ± 1806.8 | 2197 ± 1334.3 | 1067 ± 941.8 |
| Liver | 0.02 ± 0.011 | 0.04 ± 0.024 | 0.12 ± 0.022 | 0.18 ± 0.150 |
| Spleen | ND | 0.03 ± 0.009 | 0.07 ± 0.006 | 0.11 ± 0.073 |
| Kidney | ND | ND | 0.03 ± 0.008 | 0.07 ± 0.025 |
| End region of femur | 0.02^{b} | 0.11 ± 0.104 | 0.32 ± 0.068 | 1.74 ± 0.424 |
| Spine | 0.01^{b} | 0.03 ± 0.030 | 0.07 ± 0.010 | 0.45 ± 0.123 |

*% ID/g = [radioactive concentration in an organ (PSL/weight of an organ (g))/administered radioactive concentration (PSL)] × 100
Numerals indicate mean ± standard deviation (n = 3).
^{a}not detected.
^{b}mean value of two individuals (not detected in one individual)

As shown in Table 2, when rats were administered with the ¹⁶⁶Ho(NO₃)₃.5H₂O solution not containing chitosan, after 6 and 24 hrs, radioactive concentrations (% ID/g) were respectively 2-fold and 3-fold lower in the administration site, and 2 to 4-fold higher in the radioactivity-detected organs including the liver, spleen, kidney and the end region of the femur, than in the complex solution group. That is, when the radionuclide not forming a complex with chitosan was administered, only about 30% was retained in the administration site, the prostate gland. In contrast, when the radioisotope-chitosan complex of the present invention was administered, almost 90% was retained in the administration site. These results revealed that, unlike the administration of the radionuclide ¹⁶⁶Ho(NO₃)₃.5H₂O alone, the administration of the complex solution that is a recombination of the radionuclide and chitosan prevents the radioactivity from spreading to the whole body while limiting the range of the radioactivity only to an administration site.

### EXPERIMENTAL EXAMPLE 3

After the complex solution was administered, accurate radioactive concentrations were measured in various organs. 25 μl of the complex solution as a test drug were administered to the prostate gland in the abdominal region of SD rats, which was equal to about 100 μCi. After 0.5, 2, 6, 24, 72 and 144 hrs, the rats were anesthetized with diethylether and exsanguinated by incision of abdominal aorta. Various organs and tissues were collected, which included blood, plasma, brain, thymus, heart, lung, liver, kidney, adrenal gland, spleen, pancreas, testes, prostate gland (including an administration site), seminal vesicle, epididymis, bladder, skeletal muscle, bones, bone marrow, skin, carcass, stomach, small intestine, rectum and large intestine. The collected organs and tissues were weighed, sectioned and homogenized. The whole sample or the supernatant was assayed for radioactivity using a liquid scintillation counter. The measured radioactive concentrations in various organs and tissues according to the time are given in Table 3 (% ID/g) and Table 4 (% of dose).

TABLE 3
Radioactive concentrations (% ID/g) in various organs and
tissues after injection of the complex solution into prostate
gland

| | Radioactive concentrations (% ID/g [×10⁻⁴, mean ± SD]) | | | | | |
|---|---|---|---|---|---|---|
| Tissues | 30 min | 2 hrs | 6 hrs | 24 hrs | 72 hrs | 144 hrs |
| Blood | 26 ± 10 | 19 ± 9 | ND | ND | ND | ND |
| Plasma | 44 ± 16 | 32 ± 11 | ND | ND | ND | ND |
| Brain | ND* | ND | ND | ND | ND | ND |
| Thymus | 13 ± 4 | ND | ND | 34 | ND | ND |
| Heart | 15 ± 4 | ND | ND | 19 | 29 ± 7 | ND |
| Lung | 51 ± 25 | 68 ± 29 | 54 ± 27 | 28 ± 10 | 45 ± 17 | ND |
| Liver | 134 ± 59 | 268 ± 71 | 208 ± 107 | 394 ± 97 | 350 ± 118 | 243 ± 115 |
| Kidney | 40 ± 20 | 50 ± 18 | 42 | 85 ± 37 | 215 ± 78 | 224 ± 75 |
| Adrenal | ND | ND | ND | ND | ND | ND |
| Spleen | 69 ± 43 | 151 ± 58 | 107 ± 65 | 343 ± 176 | 335 ± 86 | 239 ± 110 |
| Pancreas | 46 ± 33 | 57 ± 7 | 39 | 107 ± 61 | 167 ± 68 | ND |
| Testes | ND | 88 | ND | ND | ND | ND |
| Prostate | 5461290 ± 733188 | 4907055 ± 664832 | 4566012 ± 1337741 | 5321329 ± 1153873 | 2972396 ± 312343 | 3051762 ± 664511 |
| gland | | | | | | |
| Seminal | 2077 ± 1699 | 852 ± 733 | 702 ± 145 | 531 ± 297 | 1515 ± 1304 | 1455 ± 1914 |
| vesicle | | | | | | |
| Epididymis | 65 ± 77 | 29 ± 12 | ND | 27 | 86 ± 37 | ND |
| Bladder | 2893 ± 3997 | 2955 ± 856 | 4301 ± 2101 | 3365 ± 3621 | 3592 ± 3325 | 2445 ± 3357 |
| Skeletal | ND | ND | ND | ND | ND | ND |
| muscle | | | | | | |
| Bones | 71 ± 55 | 189 ± 79 | 185 ± 107 | 711 ± 413 | 2343 ± 827 | 1708 ± 506 |
| Bone marrow | ND | ND | 17 | 32 ± 4 | 109 ± 40 | ND |
| Skin | ND | ND | ND | ND | ND | ND |
| Carcass | 17 ± 11 | 67 ± 10 | 40 ± 12 | 75 ± 18 | 143 ± 43 | 161 |
| Stomach | 71 ± 60 | 27 | ND | 46 ± 22 | ND | ND |
| Small | 52 ± 47 | 49 ± 10 | 24 | 26 ± 10 | 29 ± 6 | ND |
| intestine | | | | | | |
| Large intestine | 35 | 19 ± 9 | 23 | 50 | 34 | ND |
| Rectum | 129 ± 38 | 90 ± 50 | ND | 67 ± 27 | 70 ± 59 | ND |

% ID/g = [radioactive concentration in tissue (dpm/tissue weight (g or ml))/administered radioactive concentration (dpm)] × 100
*not detected.

TABLE 4
Radioactive concentrations (% of dose) in various organs and
tissues after injection of the complex solution into prostate
gland

| | Radioactive concentrations (% of Dose [mean ± SD]) | | | | | |
|---|---|---|---|---|---|---|
| Tissues | 30 min | 2 hrs | 6 hrs | 24 hrs | 72 hrs | 144 hrs |
| Blood | —^{a} | — | — | — | — | — |
| Plasma | — | — | — | — | — | — |
| Brain | ND* | ND | ND | ND | ND | ND |
| Thymus | 0.00 ± 0.00 | ND | ND | 0.00 | ND | ND |
| Heart | 0.00 ± 0.00 | ND | ND | 0.00 | 0.00 ± 0.00 | ND |
| Lung | 0.01 ± 0.01 | 0.01 ± 0.01 | 0.01 | 0.00 ± 0.00 | 0.00 ± 0.01 | ND |
| Liver | 0.12 ± 0.05 | 0.25 ± 0.06 | 0.20 ± 0.10 | 0.36 ± 0.07 | 0.36 ± 0.12 | 0.30 ± 0.13 |
| Kidney | 0.01 ± 0.01 | 0.01 ± 0.000 | 0.01 | 0.01 ± 0.01 | 0.04 ± 0.02 | 0.05 ± 0.02 |
| Adrenal | ND | ND | ND | ND | ND | ND |
| Spleen | 0.01 ± 0.01 | 0.01 ± 0.01 | 0.01 ± 0.01 | 0.02 ± 0.01 | 0.02 ± 0.01 | 0.02 ± 0.01 |
| Pancreas | 0.01 ± 0.01 | 0.00 ± 0.01 | 0.00 | 0.01 ± 0.01 | 0.02 ± 0.01 | ND |
| Testes | ND | 0.02 | ND | ND | ND | ND |
| Prostate | 100.21 ± 2.28 | 99.23 ± 4.77 | 98.50 ± 2.18 | 99.28 ± 1.48 | 98.84 ± 1.84 | 98.53 ± 1.84 |
| gland | | | | | | |
| Seminal | 0.05 ± 0.04 | 0.02 ± 0.02 | 0.02 | 0.01 ± 0.01 | 0.04 ± 0.04 | 0.04 ± 0.05 |
| vesicle | | | | | | |
| Epididymis | 0.00 ± 0.00 | 0.00 ± 0.00 | ND | 0.00 | 0.00 ± 0.00 | ND |
| Bladder | 0.04 ± 0.04 | 0.03 ± 0.01 | 0.05 ± 0.03 | 0.03 ± 0.03 | 0.04 ± 0.04 | 0.02 ± 0.03 |
| Skeletal | — | — | — | — | — | — |
| muscle | | | | | | |
| Bones | — | — | — | — | — | — |
| Bone marrow | — | — | — | — | — | — |
| Skin | ND | ND | ND | ND | ND | ND |
| Carcass | 0.19 ± 0.12 | 0.78 ± 0.12 | 0.48 ± 0.17 | 0.87 ± 0.16 | 1.72 ± 0.47 | 2.28 |
| Stomach | 0.02 ± 0.02 | 0.01 ± 0.00 | ND | 0.01 ± 0.01 | ND | ND |
| Small | 0.05 ± 0.05 | 0.05 ± 0.01 | 0.03 | 0.03 ± 0.01 | 0.03 ± 0.01 | ND |
| intestine | | | | | | |
| Large | 0.03 | 0.01 ± 0.01 | 0.02 | 0.04 ± 0.01 | 0.03 | ND |
| intestine | | | | | | |
| Rectum | 0.00 ± 0.00 | 0.00 ± 0.00 | ND | 0.00 ± 0.00 | 0.00 ± 0.00 | ND |

% of dose = [radioactive concentration in tissue (dpm)/administered radioactive concentration (dpm)] × 100
*not detected.
^{a}a radioactive concentration for a whole organ and tissue was not identified because only a portion of a tissue was used for radioactivity measurement

30 min after administration of the complex solution, high concentration radioactivity was detected in the administration site, the prostate gland, and over 98% of administered radioactivity was retained in the administration site until 144 hrs. Radioactivity in most tissues except for the administration site and adjacent organs were detected in low concentrations, but slowly increased along with time. 72 hrs after administration, among organs other than the prostate gland, the highest radioactive concentration was found in carcass (1.72%) and the liver (0.36%), whereas other tissues exhibited a radioactive concentration of less than 0.1%. 144 hrs after administration, radioactivity decreased in all tissues except for the kidney and the carcass. These results indicate that, when the present complex is administered to the prostate gland, it rarely spreads to the whole body. That is, these results indicate that the administered radioisotope-chitosan complex is deposited in the prostate gland, and its radioactivity is rarely irradiated to surrounding organs.

### EXPERIMENTAL EXAMPLE 4

After the complex solution was administered to the prostate gland, cumulative urinary and fecal excretions of radioactivity were examined to investigate the amount of excreted ¹⁶⁶Ho. 25 μl of the test drug was administered to the prostate gland of SD rats, which was equal to about 100 μCi. The amount of radioactivity excreted for 72 hrs was measured. The rats were kept in metabolic cages made of a stainless material. Urine and feces were collected, and, 72 hrs after administration, the rats were sacrificed with diethylether. Radioactivity remaining in the carcass and the prostate gland was also measured. The results are given in Table 5, below.

TABLE 5
Excreted radioactivity for 72 hrs after the complex solution
is administered to the prostate gland

| | Observation period | Cumulative excretions (% of dose) | | | | |
|---|---|---|---|---|---|---|
| Samples | (hr) | 1 | 2 | 3 | Mean | SD |
| Urine | 0-3 | 0.03 | 0.03 | 0.04 | 0.03 | 0.01 |
| | 0-6 | 0.04 | 0.04 | 0.05 | 0.04 | 0.01 |
| | 0-24 | 0.11 | 0.13 | 0.17 | 0.14 | 0.03 |
| | 0-48 | 0.21 | 0.25 | 0.30 | 0.25 | 0.05 |
| | 0-72 | 0.28 | 0.36 | 0.41 | 0.35 | 0.07 |
| Feces | 0-24 | 0.03 | 0.03 | 0.01 | 0.02 | 0.01 |
| | 0-48 | 0.07 | 0.07 | 0.05 | 0.06 | 0.01 |
| | 0-72 | 0.11 | 0.12 | 0.11 | 0.11 | 0.01 |
| Carcass | 72 | 2.32 | 3.27 | 2.42 | 2.67 | 0.52 |
| Prostate | 72 | 98.84 | 96.79 | 101.94 | 99.19 | 2.59 |
| gland | | | | | | |
| Total | | 99.23 | 97.27 | 102.46 | 99.65 | 2.62 |
| recovery | | | | | | |
| rate (%) | | | | | | |

At 72 hrs, percentages of cumulative radioactivity excretion in the urine and feces were 0.35% and 0.11% of dose, respectively, and were very little. The percentage of radioactive remains in the carcass was 2.67% and that in the prostate gland was 99.19%. Those data indicate that most of the dose still existed in the prostate gland at 72 hr after administration.

### EXPERIMENTAL EXAMPLE 5

To investigate the effect of the complex on hormone-independent prostate cancer, a DU-145 carcinoma cell line (hormone-independent prostate carcinoma cell line), which is rarely produces prostate-specific antigen, was transplanted into nude mice and the antitumor effect of the complex was evaluated.

The DU-145 cells were cultured in 10% FBS (fetal bovine serum)-containing F-12K(Kaign's Modification of Ham's F-12 medium) in a 5% CO₂ incubator at 37° C., and 2×10⁵ cells/0.2 ml/head were subcutaneously transplanted into the lumbar portion of male 6-week nude mice. When the long axis of the tumor was about 1 cm long, the complex solution was intratumorally injected. The administration volume of the test drug was 0.2 ml. The mice were divided into four groups: a control group (no treatment); two administration groups administered with the complex solution at doses of 10 mCi and 20 mCi per 1 cm³ tumor; and another administration group administered with a non-radioactive complex solution [Holmium-165 (¹⁶⁵Ho)-chitosan complex solution]. Two weeks after administration with the complex solution, the mice were histopathologically examined to evaluate the antitumor effect and toxicity to major organs of the complex. Changes in tumor volume after administration of the complex solution are given in Table 6.

TABLE 6
Changes in DU-145 tumor volume after administration of the
complex solution

| | Tumor volume after | | |
|---|---|---|---|
| | injection of the complex | | Tumor growth |
| | (cm³) | | inhibition rate |
| Test groups | 1 wk | 2 wks | (%) |
| Control | 1.67 ± 0.81 | 6.54 ± 1.33 | — |
| Non-radioactive | 1.24 ± 0.61 | 5.03 ± 1.51 | 23.1 |
| complex | | | |
| Complex administered | | | |
| group | | | |
| 10 mCi | 0.48 ± 0.35 | 0.32 ± 0.20* | 95.1 |
| 20 mCi | 0.28 ± 0.04 | 0.09 ± 0.24* | 98.6 |

Tumor growth inhibition rate (%) = [1 − [(average tumor volume of test group on the last day of the test, cm³)/(average tumor volume of control group on the last day of the test, cm³)]] × 100
*P < 0.05 (compared to the control group)

As a result of the tumor volumes measured the last day after administration of the complex solution, the 10 mCi and 20 mCi administration groups had tumor growth inhibition rates of 95.1% and 98. 6%, respectively. In contrast, tumor volumes were greatly increased in the control group and the non-radioactive complex administration group.

### EXPERIMENTAL EXAMPLE 6

To evaluate the effect of the complex on hormone-independent prostate cancer, an AIT carcinoma (androgen-independent carcinoma cell line) was transplanted into noble rats, and the antitumor effect of the complex was examined.

An androgen-independent, prostate carcinoma cell line, AIT, which is derived from noble rats, was cultured in 10% FBS-containing DMEM at 37° under 5% CO₂. The cultured carcinoma cells were suspended in sterile physiological saline at a density of 4×10⁶ cells/ml, and 2×10⁶ cells were subcutaneously transplanted into the lateral abdomen of male 5-week noble rats. When the long axis of the tumor was about 1 cm long (in this test, 14 days after transplantation), the rats were grouped according to the tumor mass. The kit prepared in Example 2 was used as a test drug, and was administered at the same dose as in Experimental Example 5, that is, 0.2 ml. The rats were divided into four groups: a control group (no treatment); two administration groups administered with the complex solution at 10 mCi and 20 mCi per 1 cm³ tumor; and another administration group administered with a non-radioactive complex solution (Holmium-165 (¹⁶⁵Ho)-chitosan complex solution). Four weeks after administration with the complex solution, tumors were excised from the rats, and tumor growth inhibition rates were measured by weighing the tumors. The results are given in Table 7.

TABLE 7
Inhibitory effect of the complex against the tumor growth in
the AIT subcutaneous tumor model

| | | Tumor growth |
|---|---|---|
| | Tumor | inhibition rate |
| Test groups | weight(g)^{a} | (%) |
| Control | 22.7 ± 2.05 | — |
| Non-radioactive complex | 18.1 ± 4.37 | 20.4 |
| Complex administered | | |
| group | | |
| 10 mCi | 2.1 ± 0.78* | 90.7 |
| 20 mCi | 0.7 ± 0.18* | 96.7 |

Tumor growth inhibition rate (%) = [1 − [(average tumor weight of test group on the last day of the test, g)/(average tumor weight of control group on the last day of the test, g)]] × 100
^{a}each numeral indicate mean ± SD (n = 10)
*P < 0.05 (compared to the control group)

Subcutaneously transplanted AIT tumors were excised on the last day of the test after administration. As a result, similar to the DU-145-transplanted nude mice, the complex solution-administered rats were found to have a high tumor growth inhibition rate of higher than 90%.

On the other hand, when administered with the non-radioactive complex solution, both DU-145 and AIT-transplanted models exhibited a tumor growth inhibition rate of about 20% compared to the tumor volume and weight of the control group. These results are believed to result from the non-radioactive complex solution (holmium-165 (¹⁶⁵Ho)-chitosan complex solution) interrupting the blood flow in the tumor as it becomes a gel.

### EXPERIMENTAL EXAMPLE 7

In addition to the subcutaneous tumor model tested in Experimental Example 6, the antitumor effect and side effects of the complex solution was examined in an orthotopic tumor model where AIT prostate carcinoma cells had been directly transplanted into the prostate gland. AIT cells were cultured in 10% FBS-containing DMEM at 37° under 5% CO₂. The cultured carcinoma cells were suspended in sterile physiological saline at a density of 2×10⁶ cells/0.05 ml. After noble rats were anesthetized with fentanyl and the abdomen was opened, 2×10⁶ cells per rat were transplanted into the prostate gland of the rats. Seven days after carcinoma transplantation, the rats were randomly allocated. The kit prepared in Example 2 was used as a test drug, and 0.05 ml of the kit was administered. The rats were divided into four groups: a control group (no treatment); two administration groups administered with 0.5 mCi and 1.0 mCi of the complex solution; and another administration group administered with a non-radioactive complex solution (Holmium-165 (¹⁶⁵Ho)-chitosan complex solution). Two and four weeks after administration of the complex solution, the rats were sacrificed, and tumors were excised from the rats and weighed. The results are given in Table 8.

TABLE 8
Inhibitory effect of the complex against the tumor growth in
the orthotopic tumor model where AIT has been transplanted
into the prostate gland

Tumor weight (g)^{a}

| | 2 wks after | 4 wks after |
|---|---|---|
| Test groups | administration | administration |
| Control | 1.95 ± 0.33 | 31.80 ± 6.72 |
| Non-radioactive complex | 1.93 ± 0.29 | 32.62 ± 2.32 |
| Complex administered | | |
| group | | |
| 0.5 mCi | 0.45 ± 0.07^{*,†} | 1.58 ± 0.97^{*,†} |
| 1.0 mCi | 0.49 ± 0.08^{*,†} | 1.09 ± 0.32^{*,†} |

^{a}each numeral indicate mean ± SD (n = 10)
*P < 0.05 compared to the control group
^{†}P < 0.05 compared to the non-radioactive complex administered group

Two weeks after administration with the complex, the present complex showed an inhibitory effect against the tumor growth. Four weeks after administration, in a manner similar to the subcutaneous tumor model where AIT was transplanted, the present complex exhibited a tumor growth inhibition activity of over 90% compared to the control group and the non-radioactive complex administration group.

### EXPERIMENTAL EXAMPLE 8

From all rats tested in Experimental Example 7, which were sacrificed on the last day of the test, the heart, lung, liver, kidney, spleen, testes, epididymis, seminal vesicle, bladder, rectum and tumor tissues were excised, embedded in paraffin according to general tissue processing, and sectioned into a thickness of 3 μm. The tissue sections were subjected to double staining with hematoxylin and eosin and histopathologically examined.

As a result, like the control group and the non-radioactive complex administration group, no abnormalities in adjacent organs were found in the group administered with the present complex solution (FIG. 1). In contrast, in the prostate gland (tumor tissue) of the complex solution administration group, a large necrosis was found at the center of the complex solution-administered site (the C and D of FIG. 2, the necrosis indicated by an arrow), and this tumor cell necrosis was more obvious compared to the tumor tissues of the control group (the A and B of FIG. 2). These results indicate that the present complex solution induces the necrosis of cancer cells by locally acting only on the tumor in an administration site.

### INDUSTRIAL APPLICABILITY

As described hereinbefore, the present invention provides a radioisotope-chitosan complex for treating prostate cancer. When directly administered to a prostate cancer tissue, the radioisotope-chitosan complex is deposited in the applied target site while not leaking from the applied target site, and strongly inhibits the growth of prostate cancer cells while minimizing the side effects of the conventional therapies, including urinary incontinence, urethral stricture and rectal bleeding. In addition, the radioisotope-chitosan complex may be used as an effective therapeutic agent for hormone-independent prostate cancer that is resistant to hormone therapy.

1. A composition for treating prostate cancer, comprising as an effective ingredient a radioisotope-chitosan complex that includes a therapeutic radioisotope emitting beta radiation and a chitosan.

2. The composition for treating prostate cancer according to claim 1, wherein the therapeutic radioisotope emits both high-energy beta radiation and low-energy gamma radiation.

3. The composition for treating prostate cancer according to claim 2, wherein the therapeutic radioisotope is a therapeutic radionuclide selected from the group consisting of ¹⁵³Sm, ¹⁶⁵Dy, ¹⁶⁶Ho and ¹⁶⁹Er, and the chitosan has a molecular weight of 100,000 to 1,500,000.

4. The composition for treating prostate cancer according to claim 3, wherein the therapeutic radionuclide is ¹⁶⁶Ho, and the chitosan has a molecular weight of 400,000 to 1,300,000.

5. The composition for treating prostate cancer according to any one of claims 1 to 4, wherein the composition is used for internal radiation therapy.

6. A kit for preparing the composition according to claim 1, comprising:
a reagent-A containing a radioisotope; and
a reagent-B containing a chitosan.

7. The kit according to claim 6, wherein the radioisotope is an oxide, a nitrate or a chloride of a therapeutic radionuclide that emits both high-energy beta radiation and low-energy gamma radiation.

8. The kit according to claim 6, wherein the radioisotope is an oxide, a nitrate or a chloride of a therapeutic radionuclide that is selected from the group consisting of ¹⁵³ Sm, ¹⁶⁵ Dy, ¹⁶⁶Ho and ¹⁶⁹Er.

9. The kit according to any one of claims 6 to 8, wherein the radioisotope is provided in an aqueous solution.

10. The kit according to claim 9, wherein the radioisotope in the reagent-A has a final concentration of 0.5-150 mCi.

11. The kit according to claim 6, wherein the chitosan has a molecular weight of 400,000 to 1,300,000.

12. The kit according to claims 6 or 11, wherein the chitosan is dissolved in a weak acid solution.

13. The kit according to claim 12, wherein the chitosan comprises one or more selected from the group consisting of a pH controller, an isotonic adjusting agent, a preservative and a stabilizer.

14. The kit according to claims 6 or 11, wherein the chitosan is dissolved in a weak acid solution and freeze-dried.
